(19) United States
(12) Patent Application Publication
(10) Pub. No.: US 2022/0370743 A1
(43) Pub. Date: Nov. 24, 2022

(54) AUTOMATIC CONTROL SYSTEM FOR MANUAL MECHANICAL VENTILATION DEVICE

(71) Applicant: GPA Medical, S.L., Barcelona (ES)

(72) Inventors: Pau ROMAGOSA CALATAYUD, Barcelona (ES); Pau SARSANEDAS MILLET, Barcelona (ES); Miguel Francisco PEREZ PLANAS, Barcelona (ES); Karim BOUFARESS BOUFARESS, Barcelona (ES)

(21) Appl. No.: 17/749,589

(22) Filed: May 20, 2022

(30) Foreign Application Priority Data
May 20, 2021 (EP) 21382457.6

(51) Int. Cl. A61M 16/00; A61M 16/10

(52) U.S. Cl. CPC A61M 16/0063; A61M 16/1055; A61M 16/024; A61M 16/0003; A61M 2205/3584; A61M 2016/0033; A61M 2016/0027

(57) ABSTRACT

An automatic control system for a manual mechanical ventilation device comprising a servomotor, an actuator and a stepper cylinder adapted to compress the mechanical ventilation device in response to signals sent by a controller. The controller is further connected to at least one flow sensor and one pressure sensor and has a human-machine interface for the insertion of pressure and oxygen flow output values to be received by a patient.

### TECHNICAL FIELD

This invention falls within the medical sector, specifically in the field of respiratory support devices.

### BACKGROUND

Respiratory life support devices in hospitals include respirators or mechanical ventilators to supplement lung ventilation.

In situations that overwhelm hospitals, such as epidemics and pandemics, there is a need to make up for the lack of ventilators.

For this reason, several medical devices have recently been developed for mechanical ventilation in emergency situations. Many of these products are based on the automation of a hand-held ventilator (also known by its widely used brand name, Ambu®, from “Airway Mask Bag Unit”). A resuscitator or manual respirator consists of a self-inflating air bladder connected to a mask through a set of valves. When the mask is properly applied and the chamber is manually compressed, the device forces air into the patient's lungs. When manual compression ceases, the chamber self-inflates through the other end with atmospheric or oxygen-enriched air, while allowing the lungs to empty into the environment through the valve assembly. It is a device used in medical vehicles and in transient situations in hospitals. However, they are limited for short periods of time, as they must be manually operated by healthcare personnel. In addition, the manual action required means that constant ventilation is not provided, as parameters such as breaths per minute, total volume applied, and inspiration/expiration ratio are difficult to assess subjectively.

Therefore, in the midst of a global pandemic, there is a pressing need to improve the properties of medical devices for emergency mechanical ventilation to bring the ventilation produced in the patient closer to those that would be provided by an advanced medical ventilator.

### SUMMARY

The object of the present invention is to provide a control system for manual ventilators that allows automatic ventilation to be controlled much more efficiently and accurately than prior art devices, enabling the practitioner to adapt the flow of oxygen and the pressure applied according to the needs of the patient.

To this end, the present invention comprises a system for controlling the ventilation produced in the patient by a hand-held ventilator based on the automation of the breathing process. The system is provided for this purpose with a linear actuator with servomotor that regulates the compression of the ventilator and is controlled by a programmable logic controller that receives data from at least one pressure sensor and at least one flow sensor. The system is further provided with an interface for inputting pressure and flow output values. Based on these output values and the sensor readings, the controller acts on the actuator. The components can be connected to each other via a Profinet network and are advantageously equipped with an uninterruptible power supply. A one-way valve, PEEP valve and a humidifying and/or antibacterial filter just before the patient connection can optionally be located in the patient-side socket.

The invention is intended for patients requiring mechanical ventilation or in cardiorespiratory arrest. Its use is limited to medical personnel qualified in the technique of invasive mechanical ventilation.

### BRIEF DESCRIPTION OF THE FIGURES

FIG. 1 is a schematic of the system of the invention.

FIG. 2 is a graph of the respiratory cycle and the insufflation period by mechanical ventilation controlled by the system of the invention.

FIG. 3 is a particular implementation of the control system applied to an existing ventilator.

### DETAILED DESCRIPTION

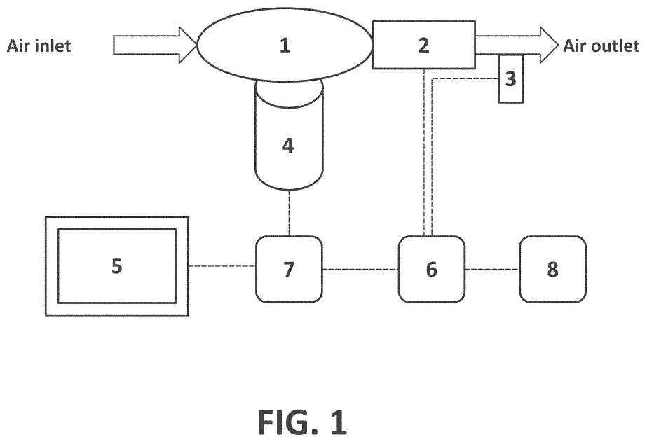

An embodiment of the control system for a hand-held ventilator according to the invention is shown in FIG. 1. The system has an air inlet to the hand-held ventilator and an air outlet from the hand-held ventilator. The control system is equipped with at least one flow sensor and at least one pressure sensor. Signals from these sensors go to the programmable logic controller, which controls the actuator that compresses the ventilation device according to the desired final parameters that the practitioner enters into the system via a human-machine interface based on the sensor readings.

The software has to control and communicate with:

Programmable Logic Controller (PLC)

Interface

Actuator

Pressure sensor

Flow sensor

Uninterruptible Power Supply (UPS).

The flow sensor reports and monitors the volume insufflated to the patient in a given time. The pressure sensor controls the pressure ranges applied to the patient. The equipment controls the ventilation applied to the patient by modulating the speed, pressure and stroke that the actuator exerts on the hand-held ventilator.

Using the interface screen, the clinician verifies and modifies the ventilator's operation.

FIG. 1 shows an embodiment of a control system for mechanical ventilation based on the automation of a hand-held ventilator (1) according to the invention. The system includes a flow sensor (2) and a pressure sensor (3). The compression of the ventilator bag is performed by a servomotor, an actuator (4) and a stepper cylinder. The device has a human-machine interface (5), or HMI, with a display that allows both the input of the necessary parameters for the patient according to the physician and the reading of the information provided by the sensors. An uninterruptible power supply (UPS) shall be used to keep the equipment operational in the absence of external power.

The essential elements of the system are:

(1) hand-held respirator

(2) flow sensor

(3) pressure sensor

(4) servomotor, actuator and stepper cylinder.

(5) human-machine interface

(6) controller

(7) switch

(8) uninterruptible (continuous) power supply system

The automatic drive part allows the translation of a cylinder step by step by means of a servomotor and actuator associated with the cylinder. The movement of the stepper cylinder in turn allows the hand-held ventilator to be compressed to deliver air to the patient.

The system may include elements to maintain the hand-held ventilator in a correct position for operation. Such elements may include, for example, a set of elastic bands.

The system shall have a flow sensor to report and monitor the volume insufflated to the patient. The insufflated volume is calculated by measuring flow and time.

The system will also have a pressure sensor to monitor the pressure ranges applied to the patient.

To control the stepper cylinder, the servo motor actuator communicates directly with the controller, which manages the displacements of the stepper cylinder.

In order to unify the communications of all devices, a Profinet network and a switch shall preferably be used. The switch does not interfere with the other devices as it performs the passive link functions to link the frames/packets between devices. The Profinet industrial network is based on TCP/IP communications but adapted for the exchange of data with industrial devices such as PLC, HMI, Servo drives, etc. . . . where the exchange of packets is done in a fast and deterministic way guaranteeing the quality of service.

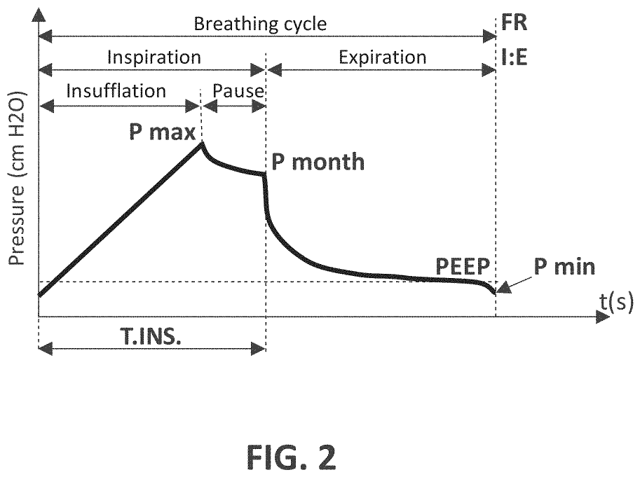

FIG. 2 shows a graph of the insufflation periods. These periods depend on the mode of operation of the system:

Volume control: mandatory ventilatory mode for sedated patients, where the tidal volume is set by the “insufflation” parameter, and respiratory cycles are controlled by respiratory rate and inspiration-expiration (I:E) ratio.

Volume assisted control: ventilatory support mode for the initial phase of weaning (known as weaning), where the patient has the possibility to set the ventilatory rate. The physician sets the tidal volume using the “insufflation” parameter, sets the baseline respiratory rate and I:E ratio, as well as the inspiratory sensitivity (the effort the patient must make to open the inspiratory valve of the ventilator) to start the respiratory cycle.

Volume assisted: ventilatory support mode for weaning or weaning, where the patient sets the ventilatory rate. The physician enters the tidal volume using the “insufflation” parameter, sets the inspiratory time and inspiratory sensitivity for the start of the respiratory cycle. In addition, the “apnoea time” can be set, which, if exceeded, the device switches to Volume Control mode.

The system works with the following control parameters:

Insufflation in % of the volume applied by the hand-held ventilator over the maximum volume of the hand-held ventilator.

Actual respiratory rate, rpm.

Inspiration:Expiration Ratio (I:E Ratio).

Positive end-expiratory pressure (PEEP) in cm H₂O.

Insufflation time, in seconds, for Volume Assisted mode

Inspiratory sensitivity for Volume Control Assisted and Volume Assisted modes

The system receives the following monitoring parameters from the mechanical ventilation equipment:

Maximum or peak pressure, cm H₂O

Plateau pressure, cm H₂O

Minimum pressure, cm H₂O

Actual respiratory rate, rpm

Ratio I:E

The information obtained by the sensors can be represented in the form of graphs:

Pressure cm H₂O

Inspiratory flow, L/min

Tidal volume, mL

The system warns with alarms in different cases:

Maximum and minimum tidal volume, mL

Maximum and minimum breathing pressure, cm H₂O

Minimum and maximum actual respiratory rate, rpm

Disconnection of power supply

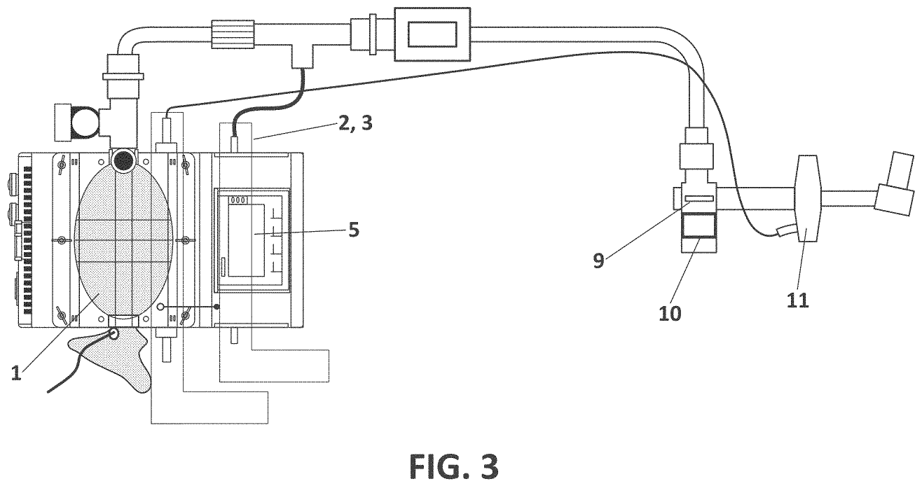

FIG. 3 shows some optional elements of the system, in addition to the previously mentioned elements. A one-way valve (9), PEEP valve (10) and a humidifying and/or antibacterial filter (11) can be found in the patient-side tubing just before the patient connection.

In view of this description and figures, the person skilled in the art will understand that the invention has been described according to some preferred embodiments thereof, but that multiple variations may be introduced in said preferred embodiments, without exceeding the subject matter of the invention as claimed.

1. An automatic control system for manual mechanical ventilation device wherein it comprises a controller and a servomotor, an actuator and a stepper cylinder adapted to compress the mechanical ventilation device in response to signals sent by the controller,
wherein said controller is connected to the servomotor and to at least one flow sensor and one pressure sensor and has a human-machine interface for the insertion of pressure and oxygen flow output values to be received by a patient,
wherein the controller is adapted to modify the compression received by the mechanical ventilation device based on the sensor input and output values entered into the interface.

2. The control system according to claim 1, wherein the controller and the sensors are connected via a profinet network and a switch.

3. The control system according to claim 1, further comprising a continuous power supply system.

4. The control system according to claim 1, wherein the interface is adapted to provide sensor readout.

5. The control system according to claim 1, further provided with an antibacterial filter on the patient side.
